(19) United States
(12) Patent Application Publication
(10) Pub. No.: US 2025/0025367 A1
(43) Pub. Date: Jan. 23, 2025

(54) NON-INVASIVE STIMULATION APPARATUS AND METHOD FOR REHABILITATION AND TREATMENT OF SEXUAL DYSFUNCTIONS

(71) Applicant: VIBE DYNAMICS PTY LTD, Canberra, Australian Capital Territory (AU)

(72) Inventors: Kym Narelle ROBINSON, Flynn (AU); Paul JENKINS, Flynn (AU)

(73) Assignee: VIBE DYNAMICS PTY LTD, Canberra, Australian Capital Territory (AU)

(21) Appl. No.: 18/848,475

(22) PCT Filed: Mar. 21, 2023
(86) PCT No.: PCT/AU2023/050203

§ 371 Date: Sep. 18, 2024

(30) Foreign Application Priority Data
Mar. 21, 2022 (AU) 2022900698

(51) Int. Cl. A61H 19/00; A61H 23/00

(52) U.S. Cl. CPC A61H 19/30; A61H 23/006; A61H 2201/10; A61H 2201/1657; A61H 2201/5007; A61H 2201/5038; A61H 2205/083; A61H 2205/087; A61H 2205/108

(57) ABSTRACT

A stimulation apparatus for use in treating sexual dysfunction, comprising: at least one pair of electrodes configured to deliver low level electrical impulses to muscles within at least one of a hip flexor, lower abdominal and thigh muscle group; a vibrating device for placement on a sexual organ of the user; and a controller comprising a stimulation circuit, a processor and a memory, the stimulation circuit operatively coupled to the pair of electrodes and vibrating device, the processor being configured to execute instructions stored in memory to: in use, control the stimulation circuit to simultaneously actuate the vibrating device and electrodes for inducing orgasm by the user; and wherein, in use, an intensity of at least one of the vibrating device and electrical impulses are dynamically varied.

### FIELD OF INVENTION

The present invention relates to a non-invasive stimulation apparatus to promote sexual rehabilitation in users that suffer from sexual dysfunctions including anorgasmia, delayed ejaculation, and anejaculation.

### BACKGROUND OF INVENTION

Sexual dysfunctions prevent males and females alike from experiencing satisfaction during any stage of sexual activity, including erection, physical pleasure, arousal and orgasm. Sexual dysfunctions can adversely impact on a sufferer's mental health, relationship intimacy and quality of life in general.

Anorgasmia (in females), anejaculation and delayed ejaculation (in males) are sexual dysfunctions that are often associated with, but not limited to, people with spinal cord injuries, advanced multiple sclerosis, and Parkinson's disease. The dysfunction is also associated with individuals undergoing cancer treatment and medical side-effects. However, even in able bodied patients, these sexual dysfunctions are common, and often experienced associated with the fertility industry.

To treat the afore-described dysfunctions various devices have been proposed. One such device comprises a vibrating means that can be placed on a user's genitalia and which provides a constant high level of stimulation for inducing orgasm. While such vibrators may bring about orgasm, they output a high amplitude and frequency that can cause extended refractory period and neural pathway exhaustion by over stimulation. This can in turn lead to reliance on the device and will often require the user to wait a substantial period of time (often in the order of weeks) before subsequent use due to over stimulation. Further, numerous medications can interfere with the vibration response, including anti-spasm medication.

Electrostimulation devices have also been proposed to potentiate or elicit an erotic sensation that can lead to orgasm. Such systems typically employ electrodes placed adjacent the user's sexual organ and configured to deliver functional electrical stimulation at a constant high frequency for inducing orgasm. However, such systems are not particularly effective for users having the afore-described sexual dysfunctions.

It would be advantageous if there was provided an apparatus and method of use which ameliorated at least one of the disadvantages of current devices as outlined above. It would also be advantageous if the apparatus facilitated rehabilitation and neuro potential, leading to a reduced reliance on the apparatus for inducing orgasm.

### SUMMARY OF INVENTION

In accordance with one aspect there is provided a stimulation apparatus for use in treating sexual dysfunction, comprising: at least one pair of electrodes configured to deliver low level electrical impulses to muscles within a hip flexor muscle group of a user and such that, when so configured, a first electrode of a first electrode pair is placed on the user's skin generally in the lower abdominal region and a second electrode of the first electrode pair is placed generally on the user's upper thigh region on the same side of the user's body as the first electrode; a vibrating device for placement on a sexual organ of the user; and a controller comprising a stimulation circuit, a processor and a memory, the stimulation circuit operatively coupled to the pair(s) of electrodes and vibrating device, the processor being configured to execute instructions stored in memory to: in use, control the stimulation circuit to simultaneously actuate the vibrating device and electrodes for inducing orgasm by the user; and wherein, in use, an intensity of at least one of the vibrating device and electrical impulses are dynamically varied during the use.

In an embodiment the electrodes and vibrating device are actuated by control signals sent from the stimulation circuit.

In an embodiment a parameter of a control signal sent to the vibrating device can be adjusted to control at least one of a vibratory frequency and amplitude for the vibrating device for dynamically controlling vibration intensity.

In an embodiment the vibratory frequency is variable between 0 and 120 Hz. The vibratory peak to peak amplitude may range between 0.25 mm and 3.00 mm.

In an embodiment a parameter of a signal sent to the electrodes can be adjusted to control at least one of an amplitude, frequency and pulse width of the electrical impulses delivered thereby for controlling intensity. The amplitude may be variable between 0 and 150 mA. The pulse width may range between 75 us to 400 us. The frequency may be variable between 0 to 80 Hz.

In an embodiment the controller is programmed to control the stimulation circuit to smoothly increase at least one of the amplitude of the electrical impulses and frequency of the vibrating output for a warm-up phase. In use, the controller may be programmed to control the stimulation circuit to smoothly increase at least one of the amplitude of the electrical impulses and frequency of the vibrating output for a climax inducing phase. The controller is programmed to control the stimulation circuit to decrease at least one of the amplitude of the electrical impulses and the frequency of the vibrating output following the warm-up phase and prior to the climax inducing phase.

The apparatus may comprise a second pair of electrodes. The electrodes in the second pair may be collaterally placed in corresponding regions to the first pair. Each electrode pair may be connected to a channel of the stimulation circuit and wherein the controller is configured to selectively actuate each channel.

In an alternative electrode placement configuration, a first electrode pair is placed on the user's skin in the lower abdominal region and a second electrode pair is placed on the user's upper thigh region on the same side of the user's body as the first electrode pair. Third and fourth electrode pairs may be collaterally placed in corresponding regions to the first and second electrode pairs.

In an embodiment at least one further electrode pair are placed on the user's lower leg for activating a tibialis anterior muscle of the user.

The electrodes may be placed anteriorly above the muscle regions.

In an embodiment at least one of a minimum and maximum intensity value of the vibrating output and electrical impulses is adjustable by the user. At least one of a timing and intensity parameter of the warm up and climax inducing phase may be adjustable by the user.

In an embodiment the memory stores one or more algorithms for dynamically controlling the vibrating output and electric impulses during use.

At least one of the algorithms may be executable for stimulating the user's muscles so as to mimic natural muscle contraction experienced by an individual who does not suffer from sexual dysfunction. In an embodiment user input for controlling the controller to manually boost at least one of an amplitude, frequency and/or pulse width of the vibrating device and electrode outputs.

In an embodiment the apparatus may further comprise a user input for pausing the stimulation applied by the electrodes while maintaining the vibration output.

In an embodiment, for a male user, the vibrating device comprises a vibrating disc for placement on or adjacent a neck of their penis.

In an embodiment, for a female user, the vibrating device comprises a vibrating disc for applying directly to their clitoris.

In an embodiment the vibrating device comprises a handle for holding the vibrating device in place.

In accordance with another aspect there is provided a stimulation apparatus for use in treating sexual dysfunction, comprising: a vibrating device for placement on a sexual organ of the user; and a controller comprising a stimulation circuit, a processor and a memory, the stimulation circuit operatively coupled to the vibrating device, the processor being configured to execute instructions stored in memory to: control the stimulation circuit to send a stimulation signal to the vibrating device for activation thereof and wherein the stimulation circuit is further configured to dynamically adjust a parameter of the stimulation signal to dynamically control at least one of a frequency and amplitude of a vibrating output based on an algorithm implemented by the controller.

In another aspect there is provided a stimulation apparatus for use in treating sexual dysfunction, comprising: electrodes configured for providing functional electrical stimulation to at least one muscle in at least one of the hip flexor, lower abdominal and thigh muscle regions; a vibrating device for placement on a sexual organ of the user; and a controller comprising a stimulation circuit, a processor and a memory, the stimulation circuit operatively coupled to the electrodes and vibrating device, the processor being configured to execute instructions stored in memory to: in use, control the stimulation circuit to simultaneously actuate the vibrating device and electrodes for inducing orgasm by the user; and wherein in use the frequency of at least one of the vibrating device and electrical impulses are dynamically varied.

In yet another aspect there is provided a stimulation apparatus for use in treating sexual dysfunction, comprising: electrodes configured for providing functional electrical stimulation to at least one muscle in at least one of the hip flexor, lower abdominal and thigh muscle regions; a controller comprising a stimulation circuit, a processor and a memory, the stimulation circuit operatively coupled to the electrodes and being configured to execute instructions stored in memory to: in use, control the stimulation circuit to actuate the electrodes for inducing orgasm by the user; and wherein in use the frequency of electrical impulses output by the electrodes are dynamically varied.

In accordance with a still further aspect there is provided a kit for use in treating sexual dysfunction, comprising: at least one pair of electrodes configured to deliver low level electrical impulses to muscles within a hip flexor muscle group of a user; a vibrating device for placement on a sexual organ of the user; and a controller comprising a stimulation circuit, a processor and a memory, the stimulation circuit operatively coupled to the pair(s) of electrodes and vibrating device and wherein, during use, the stimulation circuit is controlled to selectively actuate the vibrating device and electrodes for inducing orgasm by the user and such that the frequency of at least one of the vibrating device and electrical impulses are selectively varied by the controller during the use.

In another aspect there is provided a stimulation apparatus for use in treating sexual dysfunction, comprising: at least one pair of electrodes configured to deliver low level electrical impulses to muscles within a hip flexor muscle group of a user and such that, when so configured, a first electrode pair is placed on the user's skin in the lower abdominal region and a second electrode pair is placed on the user's upper thigh region on the same side of the user's body as the first electrode pair; a vibrating device for placement on a sexual organ of the user; and a controller comprising a stimulation circuit, a processor and a memory, the stimulation circuit operatively coupled to the pairs of electrodes and vibrating device, the processor being configured to execute instructions stored in memory to: in use, control the stimulation circuit to selectively actuate the vibrating device and electrode pairs for inducing orgasm by the user; and wherein, in use, the frequency of at least one of the vibrating device and electrical impulses are selectively varied during the use.

### BRIEF DESCRIPTION OF DRAWINGS

Embodiments of the present invention will now be described, by way of example only, with reference to the accompanying drawings, in which:

FIG. 1 is a schematic of an apparatus, in accordance with an embodiment of the present invention;

FIG. 2 is a schematic illustrating placement of electrodes on a user, in accordance with an embodiment;

FIG. 3 is a schematic showing positioning of a vibrating device and a subset of electrode arrays for a male user, in accordance with an embodiment;

FIG. 4 is a schematic showing positioning of a vibrating device and a subset of electrode arrays for a female user, in accordance with an embodiment;

FIG. 5 is a schematic showing modules implemented by a programmable controller of the apparatus of FIG. 1;

FIG. 6 is a graph showing a typical algorithmic stimulation profile implemented by the apparatus of FIG. 1 for inducing orgasm;

FIG. 7 is a graph showing a further example stimulation profile;

FIG. 8 is a schematic showing an alternative positioning of electrodes, in accordance with an embodiment; and.

FIG. 9 is a schematic showing yet another alternative positioning of electrodes, in accordance with an embodiment.

### DETAILED DESCRIPTION

Embodiments of the invention described herein relate to a stimulation apparatus for inducing climax in users who suffer from sexual dysfunction. The apparatus may advantageously promote sexual rehabilitation in females that suffer from anorgasmia and males that suffer from delayed ejaculation and anejaculation.

### Apparatus Configuration

With reference to the Figures there is shown an apparatus 10 in accordance with a first embodiment of the invention. The apparatus comprises a body 12 suitably configured for holding by a user. The body has a user friendly design making it easy to hold in one hand by those with limited hand function and/or hemiplegia. In one embodiment the body has a palm swell on one or both sides and optional anchor points for receiving a hand strap.

The body 12 houses an electronic controller 14 comprising a processor configured to implement one or more therapeutic muscle stimulation algorithms, as will be described in subsequent paragraphs. It will be understood that the term “processor” as used herein is to be construed broadly and include within its scope any device that can process the relevant inputs and outputs as described in subsequent paragraphs and may include a microprocessor, microcontroller, programmable logic device or other suitable computational device. According to the illustrated embodiment, the controller 14 is programmable by a user and as such will hereafter be referred to as a “programmable controller”.

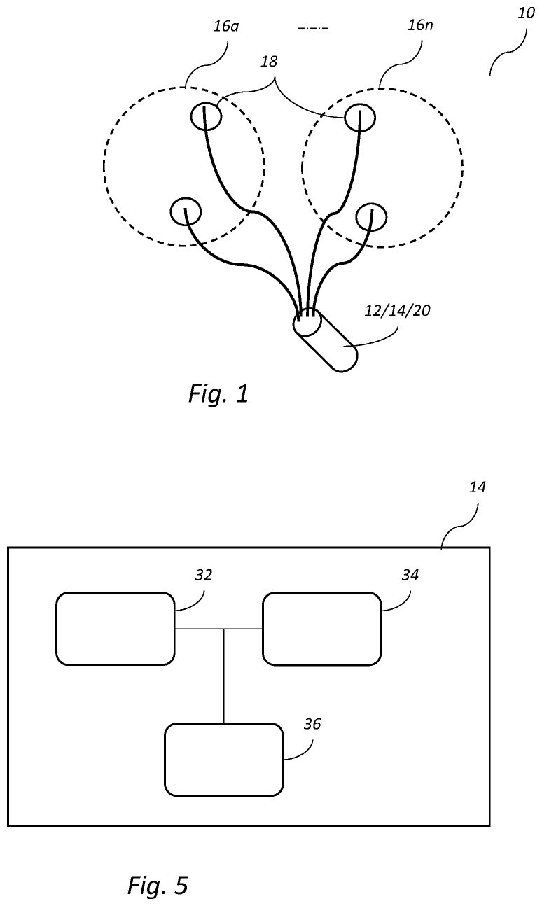

As shown in FIG. 1, the programmable controller 14 is configured to actuate electrodes 18 that are selectively placed to deliver an electrical stimulation signal for causing muscles in at least one of the hip flexors, lower abdominals, thighs and the tibialis anterior to contract. More specifically, the controller 14 is configured to apply electrical current across the surface of the skin via the electrode pairs (cathode and anode) to stimulate nerves that cause a muscular contraction in the afore-described muscles/groups. This form of muscle stimulation is generally referred to as Electrical Muscle Stimulation (EMS). Depending on the application, it is also commonly referred to as bipolar Functional Electrical Stimulation (FES) or Neuromuscular Electrical Stimulation (NMES), the latter of which is particularly designed for patients with neurological impairments and which operates to activate muscles through stimulation of intact peripheral motor nerves.

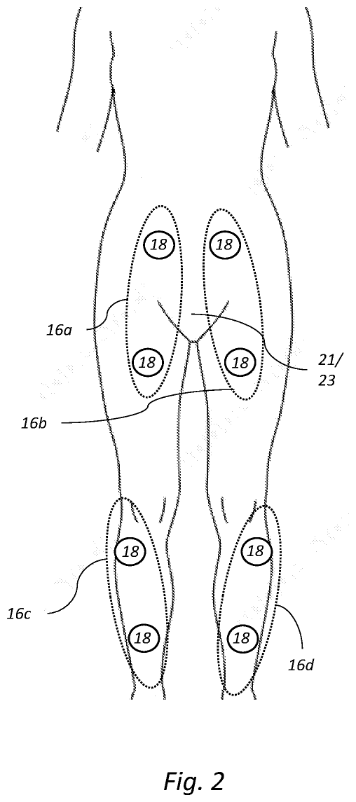

Bilateral positioning of the electrodes 18 for stimulating these four muscle groups is shown in FIG. 2. It will be understood, however, that a subset of those electrodes may be used for treatment. Furthermore, depending on the extent and type of sexual dysfunction, the electrodes 18 may need only be placed on a single side of the body (e.g., for facilitating with rehabilitation of sexual dysfunction in stroke survivors that do not have any muscle control on one side of their body).

The electrodes 18 may be attached directly to the user's skin, such as using re-usable self-adhering pads, as are well known in the art. According to the illustrated embodiment, the controller 14 implements multiple channels 16 that are each configured to stimulate a respective pair of electrodes for targeting a desired muscle grouping. According to the FIG. 2 example the controller 14 implements four channels 16a, 16b, 16c and 16d that are each connected to a corresponding electrode pair. The electrodes 18 may be spherical, cylindrical, round or may assume any of a number of different shapes and structural configurations. Thus, although the Figures show certain electrode and lead configurations, other electrode and lead configurations are possible and contemplated in the present invention.

Typically, the electrodes 18 will be placed anteriorly of the muscle group to be stimulated, thereby allowing the user to observe the underlying muscle response during treatment and identify if there are any issues or adjustments that need to be made.

When worn as shown in FIG. 2, the electrodes 18 are applied in a non-invasive and painless fashion to the user's skin. In an embodiment, the user may apply a suitably conductive gel or other suitable substance at the interface between the electrodes 18 and the skin. Body hair may need to be removed to improve electrode conductivity.

Also communicable with the programmable controller 14 is a vibrating device 20 configured to provide a vibrating/vibratory output. More particularly, the vibrating device 20 has at least one vibrating element 22 configured for stimulating a sexual organ of the user to facilitate inducement of arousal and/or orgasm.

According to the illustrated embodiment, the vibrating device 20 is incorporated into the body 12 of the apparatus 10, thus allowing it to be placed and held on, in, or adjacent the sexual organ by the user while in use. It will be understood that the vibrating device 20 may be held by a person other than the user of the apparatus 10, such as a partner.

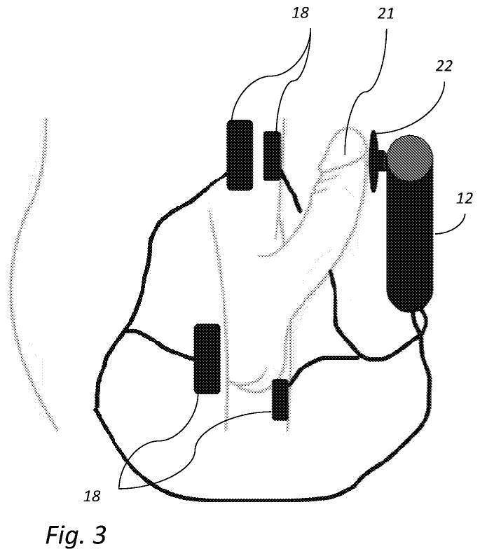

In one form, the vibrating device 20 may be particularly configured for a male user, such as shown in the example embodiment of FIG. 3. As shown, a vibrating element in the form of a disc 22 is located on the device 20 near or at one end (although it will be understood that the vibrating element could take on other forms and be disposed at any location on or in the apparatus body 12, depending on the desired configuration). The disc 22 is vibrated by way of a vibrating motor located within the body 12, using techniques well understood in the art. In use, the male user applies the vibrating disc directly to the underside of their penis 21 (i.e., their frenulum), gently sandwiching the penis 21 between device disc 22 and thumb. This may allow vibration to be applied through their hand to the dorsal nerves. It will be understood that users who have sustained damage to their frenulum may reverse the positioning and have the vibrating disc on the top side closer to the dorsal nerves.

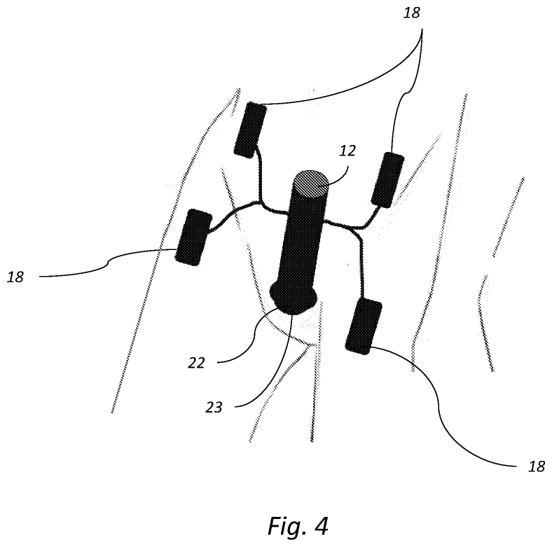

Another form of the apparatus suitable for use by a female user is shown in FIG. 4. In this case the vibrating element (again taking the form of a disc 22) is located near or at one end of the device 20 for application directly on the female user's clitoris 23. Again, it will be understood that the vibrating element could take on other forms and be disposed at other locations on or within the apparatus body 12.

It will be understood that in alternative embodiments, the vibrating device 20 may be separate to the apparatus 10. In this case, the vibrating device 20 may be remotely controlled by the programmable controller 14, e.g., via a wired or wireless link. For wireless forms of communication, the separate vibrating device 20 may include its own vibrating motor and power source, with the programmable controller 14 configured to send a control signal via a suitable wireless communications protocol. For wired coupling, a suitably configured lead may be utilised to provide signals form the programmable controller 14 that both power and provide dynamic control of the vibrating device 20.

Turning now to FIG. 5 there is shown functional components of a programmable controller 14, in accordance with an embodiment of the invention. The controller 14 comprises a stimulation circuit 32 for controlling the electrode arrays 16 and vibrating device 20. More particularly, the stimulation circuit 32 may implement an EMS pulse generator adapted for selectively pulsing the electrodes 18 via the multiple channel outputs 16a to 16n. It will be understood that the stimulation circuit 32 may be configured to simultaneously activate multiple channels, alternate activation or otherwise selectively control actuation according to the algorithms as required. The circuit 32 may further be configured as a power source for dynamically controlling the vibrating device 20. The signals output by the stimulation circuit 32 for controlling the electrodes 18 and vibrating device 20 will hereafter be referred to as “control signals”. The controller 14 also comprises a processor 34 (in this example embodiment taking the form of a microprocessor) and a memory 36. The controller 14 and its functional components may be powered by an integrated battery or external power source, depending on the desired configuration.

In more detail, the microprocessor 34 is configured to execute instructions stored in memory to control the stimulation circuit 32 to actuate the vibrating device 20 and electrodes 18 for facilitating orgasm by the user. As will be described in more detail in subsequent paragraphs, the memory 36 stores one or more therapeutic algorithms that are executable by the controller 14 to dynamically control operation of the electrodes 18 and vibrating device 20 (i.e., via the stimulation circuit 32). In this regard, the apparatus 10 may comprise a user input means (e.g., buttons, keypad, touchscreen, or the like) for generally controlling operation of the apparatus 10, including for selecting a desired algorithm (or stimulation profile). The user input means may also allow the user to turn the apparatus on and off, as well as pause operation of the electrodes 18 (while maintaining operation of the vibrating device 20) when close to climax. In addition, the user input means may include a “boost” button or other suitable user input which operates to temporarily increase a level of stimulation/vibration of one or both of the selected electrodes 18 and vibrating device 20 for inducing climax. Effectively, the boost and pause/stop buttons over-ride the therapeutic algorithms.

The controller 14 may additionally or alternatively be configured with a wired or wireless transceiver for receiving user input and new algorithms from a remote device. For example, the controller 14 may comprise a wireless transceiver (e.g., Bluetooth) for communicating with a mobile phone or other suitable electronic device having an input device (e.g., keyboard, touch screen interface, etc.) and loaded with software capable of communicating with and programming the controller 14.

### Algorithms

As stated in preceding paragraphs, the memory 36 implemented by the programmable controller 14 stores one more therapeutic algorithms. The algorithms can be thought of as mathematical functions that describe key elements of an orgasm. The programmable controller 14 is configured to dynamically control vibration and electrical muscle stimulation applying the associated mathematical functions for the user to achieve arousal and/or climax.

It will be understood that certain algorithms will be more suited to inducing orgasm in a user than others (particularly given the many varied physiological impairments that can underly the sexual dysfunction). Thus, in one form, the memory 36 may be loaded with several different predefined algorithms that a user can trial to find the one that is most effective. However, while the predefined algorithms may vary, they each operate to dynamically control various adjustable parameters of the EMS stimulation signals delivered to the electrodes 18 and the vibration control signal sent to the vibration device 20 during use. As will become evident from subsequent paragraphs, the parameters may be used to control intensity of the vibration and electrical impulses during use.

As will be described in more detail in subsequent paragraphs, according to the preferred embodiment, each of the algorithms is configured to cause the stimulation circuit 32 to implement certain predefined phases. Notably, a first phase of stimulation (referred to as the “warmup” phase) involves ramping up EMS and vibration intensity to a warmup level before reducing or completely withdrawing stimulation for a predefined period.

At least one climax inducing phase follows the warmup phase, wherein at least one the EMS and vibration intensity are increased to a higher climax inducing intensity which is maintained for a predefined period. Typically, the first climax inducing phase will ramp up the intensity to some value which is less than a predefined maximum climax intensity (i.e., for both the vibration device 20 and EMS). This may, for example, be entered by the user at setup of the apparatus and changed at any time. Determining the maximum climax value may involve the user trialling various intensities to see what feels appropriate. This may be an intensity at the upper range of their comfort level (i.e., before the stimulation becomes uncomfortable).

According to one embodiment, the stimulation circuit 32 is configured to dynamically control the frequency and/or amplitude of the vibrating output by adjusting one or more parameters of the vibration control signal (e.g., applied voltage, current, etc.) as will be understood by persons skilled in the art. In a preferred embodiment, the vibrating frequency is preferably variable between 10 and 120 Hz. Also, in a preferred embodiment the peak amplitude is preferably variable between 0.25 mm and 3.00 mm. According to the preferred embodiment, the peak amplitude is set prior to use (either by the user or algorithm) and does not change during an “in use” state of the apparatus 10. Rather, it is the frequency that dynamically varies up and down throughout the various predefined warmup, rest and climax inducing phases (examples of which are described later in further detail).

The stimulation circuit 32 is configured to adjust at least one of the following parameters of the EMS stimulation signal: amplitude (i.e., strength of applied current, measured in milliamps mA), frequency (i.e., number of electrical pulses delivered each second, measure in Hertz) and pulse width (measured in microseconds μS) of the electrical pulses. In a preferred embodiment the amplitude is variable between 1 and 150 mA, while the pulse width is variable between 75 us to 400 us and the frequency variable between 15 to 80 Hz. In a preferred embodiment, the main EMS parameter dynamically controlled during the various afore-described phases is amplitude, with the pulse width and frequency remaining generally constant (i.e., based on predefined user or settings specific to the selected algorithm).

### Example Use Case

An example use case for the apparatus 10 will now be outlined. The parameters for the algorithm described in this use case should not be seen as being limiting and are examples only.

At step S1 a user places a first electrode 18 of channel 16a is placed in the lower abdominal region on a first side of their body. The other electrode 18 of channel 16b is placed in the upper thigh region on the same side. A pair of electrodes 18 for channel 16b is then similarly placed on the other side of their body. This particular positioning of electrodes aims to specifically target the hip flexors with EMS, with spill over into the lower abdominal and upper quadriceps on each side of the body. Through extensive testing it has been found that dynamically activating these muscles/muscle groups using EMS in combination with organ stimulation (i.e., via the vibrating device 20) can greatly facilitate inducement of climax in persons with sexual dysfunctions as afore-described. In addition, the user may also place electrodes 18 above and below their tibialis anterior muscle (as shown in FIG. 2) via channels 16c and 16 for particularly targeting the peroneal nerve.

At step S2, the user holds the vibrating device 20 on their sexual organ (i.e., clitoris 23 or penis 21) as previously described and subsequently selects a therapeutic algorithm for execution by the apparatus 10. According to the illustrated example, the user has set a maximum climax amplitude of 100 mA for EMS and maximum climax frequency of 100 Hz for vibration.

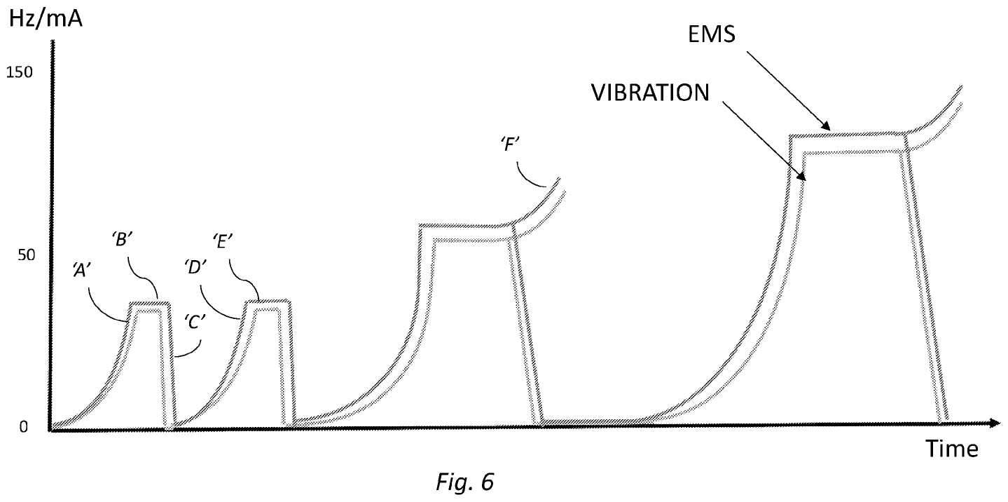

According to the illustrated example, the example algorithm initially initiates a warmup phase (step S3) in which the stimulation circuit 32 is controlled to smoothly increase the frequency of the vibration device from 0 Hz to a predefined vibrator warmup frequency, in this example being 30% of the maximum climax frequency (i.e., 30 Hz). In the same period, the controller 14 simultaneously actuates EMS channels 1 and 2 while increasing their respective amplitudes from an initial starting amplitude of 0 mA to a predefined warmup amplitude, in this example being 35% of the user's climax maximum (i.e., 35 mA). It will be understood that the warmup maximum frequency/amplitude may be different to those discussed in this example and can vary depending on the user and desired configuration. In all cases, however, the warmup value will be less than the predefined maximum climax value for both EMS and vibration. The relatively low warmup value helps the user position their body while priming for climax. For male users this can also induce a reflex erection. While an erection is not necessary to induce ejaculation or climax, it can be helpful for rehabilitation. The warmup phase is reflected by curve A and plateau B of FIG. 6. The period for this phase may vary, but according to the illustrated example is approximately 2 minutes.

The stimulation circuit 32 is then controlled by the algorithm to move to a first climax inducing phase (step S4). This involves an initial sharp reduction in the respective amplitude and frequency of the EMS and vibration signal (see line C of FIG. 6) for allowing the reflex pathway a short rest. During this rest (which according to the illustrated example is 30 seconds, although this may vary depending on the user and desired configuration), the EMS and vibrations signals are reduced to zero or some relatively low amplitude/frequency, e.g., 5 mA, 5 Hz. At the end of the rest phase, the stimulation circuit 32 smoothly increases both the EMS and vibration signals to 75% of the respective maximum climax values before plateauing for a period (see curve D and line E of FIG. 6). According to the example embodiment, the increase takes 20 seconds, with the plateau (i.e., continued high amplitude/frequency stimulation) lasting 60 seconds.

If this user hasn't reached climax during the first climax inducing phase, the EMS and vibration will drop back down to 0 and pause stimulation for a predefined time (e.g., one minute). This gives the reflex a short rest to avoid saturating the pathway. It is also an opportunity to adjust the electrodes 18 and body posture.

Depending on the algorithm, after the predefined time has elapsed (i.e., following step S4), the stimulation circuit 32 may subsequently be controlled to initiate a second climax inducing phase (step S5). During this phase, the stimulation circuit 32 may be controlled to additionally or alternatively actuate EMS channels 16c and/or 16d. The amplitude of EMS for the activated channels, in addition to the vibration device 20, are both smoothly increased from 0 to 90% of the individual's maximum climax value before plateauing. This is shown as the third ramping profile in FIG. 6.

At any time (i.e., during any of the phases), the user can engage the boost button which increases the respective amplitude and frequency values to their preset maximums to further induce climax (represented by curve F of FIG. 6).

At any stage, once climax starts, the user can turn off the EMS as their muscles will be triggered to spasm by climax (i.e., using the input means as afore-described).

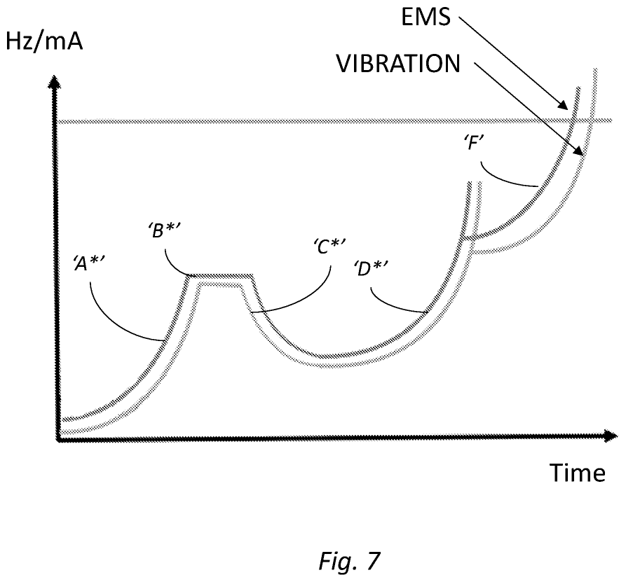

It will be understood that the above use case is simply an example of the parameters and stimulation sequence that may be implemented by an algorithm of the apparatus 10. For example, a female with a spinal cord injury may require lower amplitude vibration however the EMS parameters will remain similar to those described in the above example. Conversely, someone with multiple sclerosis represent a different use case, may typically require a lower amplitude for EMS. Someone suffering from severe sciatica may be able to tolerate a higher level but not quite as high as those with a complete spinal cord injury. There is also those with stroke/hemiplegia who will only require half the EMS channels. For those without chronic health conditions, there is the option to only use the vibration, or vibration with very low EMS. By way of example, a ramping profile for a different algorithm to that described above is shown in FIG. 7 (where A*, B*, C* and D* correspond to similar features of FIG. 6). In this case, the user has hit the boost button before the apparatus has plateaued out at the first maximum climax inducing value.

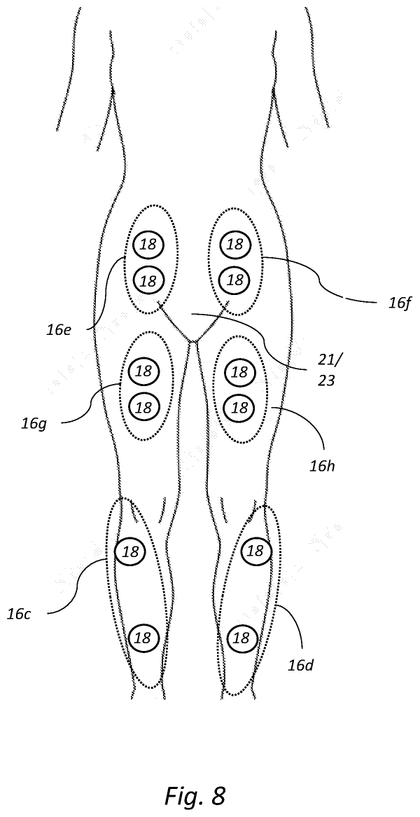

FIG. 8 depicts an alternative electrode placement configuration for activating the hip flexors during use. In this case, two pairs of electrodes (16e, 16f) are placed in the lower abdominal region (i.e., one on either side of the user's body). Similarly, a further two pairs of electrodes (16g, 16h) are placed in the upper thigh region (again, one on either side of the user's body). The respective pairs of electrodes (16e, 16f, 16g, 16h) are each actuated during user for stimulating the hip flexors during use, in much the same manner as for the electrode pairs 16a, 16b of FIG. 2, It will be understood that the pulsing of the respective pairs of electrodes (16e, 16f, 16g, 16h) may either be synchronous or asynchronous, depending on the algorithm being implemented.

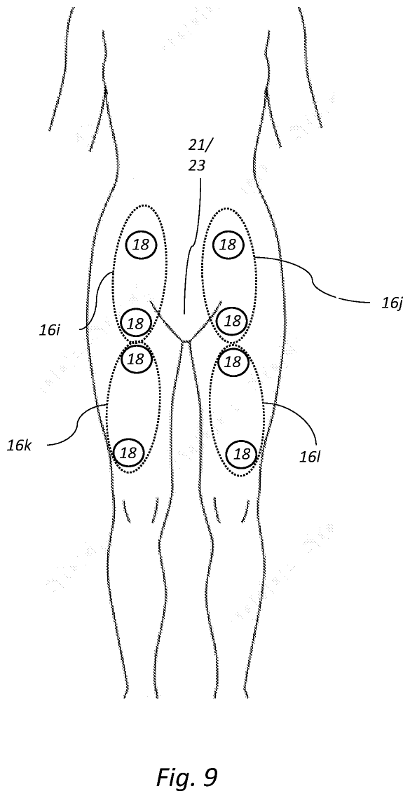

FIG. 9 depicts yet another alternative electrode placement configuration for activating the hip flexors during use. In this case, a first electrode pair 16i has one electrode 18 positioned in the lower abdominal and another electrode 18 in the upper thigh region. A second electrode pair 16j is collaterally positioned. A third electrode pair 16k on the same side as the first electrode pair 16i has a first electrode 18 positioned in the upper thigh immediately adjacent the thigh electrode 18 of the first pair 16i, while a second electrode 18 is positioned slightly above the knee. A fourth electrode pair 16l is collaterally positioned to the third electrode pair 16k. Again, the respective pairs of electrodes (16i, 16j, 16k, 16l) are each actuated during use for stimulating the hip flexors, in much the same manner as for the electrode pairs 16a, 16b of FIG. 2, It will be understood that the pulsing of the respective pairs of electrodes (16i, 16j, 16k, 16l) may either be synchronous or asynchronous, depending on the algorithm being implemented.

According to a particular embodiment, the programmable controller 14 may be controlled to gradually decrease the maximum vibrator and EMS climax values as the apparatus 10 is used to assist with rehabilitation and reduce dependency.

It will be understood that there are various chronic medical conditions that may result in sexual dysfunction, and as such it may be that the apparatus could be used exclusively with the vibrator 20 (i.e., without any EMS and with the vibrator being dynamically controlled to implement the various warm up and climax inducing stages in isolation), or vice versa with only EMS stimulation implemented by the apparatus (again with the dynamic control as afore-described). It will be understood that the EMS only stimulation implemented by the apparatus could be used in conjunction with another type of genital stimulation (such as partner directed stimulation).

It will also be understood that the while afore described example and figures showed the vibrator frequency and EMS amplitude increase and decrease in unison, they need not identically follow one another. For example, in one embodiment, there may be a deliberate lag between the increase and decrease in amplitude/frequency between the two devices (i.e., EMS and vibrator). In another form, the amplitude/frequency of one device may begin to ramp down while the other is still ramping up. The variances are within the purview of the invention and may depend on the specific algorithm being implemented.

Various advantages arise through use of an apparatus according to one or more of the afore-described embodiments, including:
Increased likelihood of climax for patients with varied sexual dysfunctions.
Non-invasive and easy to use;
Relatively short recovery time due to use of less concentrated stimulation (e.g., vibration frequency and EMS amplitude) as required by conventional devices.
Fertility benefits and option to collect semen samples at home and without medical assistance.
Increased quality of life, relationship intimacy and mental health.
Reduced pain and spasms.
Can be used to rehabilitate sexual function and increase neuro plasticity.
Can be used independently or with intimate partner assistance.
Customizable to individual needs
Can be adjusted to decrease dependence and rehabilitate client away from device if needed.

In this specification, the word “comprising” is to be understood in its “open” sense, that is, in the sense of “including”, and thus not limited to its “closed” sense, that is the sense of “consisting only of”. A corresponding meaning is to be attributed to the corresponding words “comprise”, “comprised” and “comprises” where they appear.

The preceding description is provided in relation to several embodiments which may share common characteristics and features. It is to be understood that one or more features of any one embodiment may be combinable with one or more features of the other embodiments. In addition, any single feature or combination of features in any of the embodiments may constitute additional embodiments.

In addition, the foregoing describes only some embodiments of the inventions, and alterations, modifications, additions and/or changes can be made thereto without departing from the scope and spirit of the disclosed embodiments, the embodiments being illustrative and not restrictive.

Furthermore, the inventions have described in connection with what are presently considered to be the most practical and preferred embodiments, it is to be understood that the invention is not to be limited to the disclosed embodiments, but on the contrary, is intended to cover various modifications and equivalent arrangements included within the spirit and scope of the inventions. Also, the various embodiments described above may be implemented in conjunction with other embodiments, e.g., aspects of one embodiment may be combined with aspects of another embodiment to realize yet other embodiments.

Further, each independent feature or component of any given assembly may constitute an additional embodiment.

1-30. (canceled)

31. A stimulation apparatus for use in treating sexual dysfunction, comprising:
at least one pair of electrodes configured to deliver low level electrical impulses to muscles within a hip flexor muscle group of a user and such that, when so configured, a first electrode of a first electrode pair is placed on the user's skin generally in the lower abdominal region and a second electrode of the first electrode pair is placed generally on the user's upper thigh region on the same side of the user's body as the first electrode;
a vibrating device for placement on a sexual organ of the user; and
a controller comprising a stimulation circuit, a processor and a memory, the stimulation circuit operatively coupled to the pair(s) of electrodes and vibrating device, the processor being configured to execute instructions stored in memory to:
in use, control the stimulation circuit to simultaneously actuate the vibrating device and electrodes for inducing sexual climax by the user; and wherein, in use, an intensity of at least one of the vibrating device and electrical impulses are dynamically varied during the use.

32. A stimulation apparatus in accordance with claim 31, wherein a parameter of a control signal sent to the vibrating device can be adjusted to control at least one of a vibratory frequency and amplitude for the vibrating device for dynamically controlling vibration intensity.

33. A stimulation apparatus in accordance with claim 32, wherein the vibratory frequency is variable between 0 and 120 Hz.

34. A stimulation apparatus in accordance with claim 32, wherein a peak to peak amplitude of the vibrating device ranges between 0.25 mm and 3.00 mm.

35. A stimulation apparatus in accordance with claim 31, wherein a parameter of a signal sent to the electrodes can be adjusted to control at least one of an amplitude, frequency and pulse width of the electrical impulses delivered thereby for controlling intensity.

36. A stimulation apparatus in accordance with claim 35, wherein the amplitude is variable between 0 and 150 mA.

37. A stimulation apparatus in accordance with claim 35, wherein the pulse width is variable between 75 us to 400 us.

38. A stimulation apparatus in accordance with claim 35, wherein the frequency is variable between 0 to 80 Hz.

39. A stimulation apparatus in accordance with claim 31, wherein the apparatus comprises a second pair of electrodes and wherein electrodes in the second pair are collaterally placed in corresponding regions to the first electrode pair and wherein each electrode pair is connected to a channel of the stimulation circuit and wherein the controller is configured to selectively actuate each channel.

40. A stimulation apparatus in accordance with claim 39, wherein at least one further electrode pair are placed on the user's lower leg for activating a tibialis anterior muscle of the user.

41. A stimulation apparatus in accordance with claim 39, wherein the electrodes are placed anteriorly above the muscle regions to be stimulated.

42. A stimulation apparatus in accordance with claim 31, wherein at least one of a minimum and maximum intensity value of the vibrating output and electrical impulses is adjustable by the user.

43. A stimulation apparatus in accordance with claim 31, wherein at least one of a timing and intensity parameter of a warm up and climax inducing phase implemented by the controller are adjustable by the user.

44. A stimulation apparatus in accordance with claim 31, wherein the memory stores one or more algorithms for dynamically controlling the vibrating device and electric impulses during use and wherein at least one of the algorithms is executable for stimulating the user's muscles so as to mimic natural muscle contraction experienced by an individual who does not suffer from sexual dysfunction.

45. A stimulation apparatus in accordance with claim 31, further comprising a user input for controlling the controller to perform at least one of the following functions: (a) manually boost at least one of an amplitude, frequency and/or pulse width of the vibrating device and electrode outputs; and (b) pausing the stimulation applied by the electrodes while maintaining the vibration output.

46. A stimulation apparatus in accordance with claim 31, wherein, for a male user, the vibrating device comprises a vibrating disc for placement on or adjacent a neck of their penis.

47. A stimulation apparatus in accordance with claim 31, wherein, for a female user, the vibrating device comprises a vibrating disc for applying directly to their clitoris.

48. A stimulation apparatus for use in treating sexual dysfunction, comprising:
electrodes configured for providing functional electrical stimulation to at least one muscle in at least one of the hip flexor, lower abdominal and thigh muscle regions;
a controller comprising a stimulation circuit, a processor and a memory, the stimulation circuit operatively coupled to the electrodes and being configured to execute instructions stored in memory to:
in use, control the stimulation circuit to actuate the electrodes for inducing sexual climax by the user; and wherein in use in use the amplitude and/or frequency of at least one of a vibratory output and electrical stimulation are dynamically varied.

49. A stimulation apparatus in accordance with claim 48, further comprising a vibrating device for placement on a sexual organ of the user, the stimulation further operatively coupled to the vibrating device;
wherein, in use, the stimulation circuit is further controlled to simultaneously actuate the vibrating device and electrodes for inducing sexual climax by the user.

50. A stimulation apparatus for use in treating sexual dysfunction, comprising:
at least one pair of electrodes configured to deliver low level electrical impulses to muscles within a hip flexor muscle group of a user and such that, when so configured, a first electrode pair is placed on the user's skin in the lower abdominal region and a second electrode pair is placed on the user's upper thigh region on the same side of the user's body as the first electrode pair;
a vibrating device for placement on a sexual organ of the user; and
a controller comprising a stimulation circuit, a processor and a memory, the stimulation circuit operatively coupled to the pairs of electrodes and vibrating device, the processor being configured to execute instructions stored in memory to:
in use, control the stimulation circuit to selectively actuate the vibrating device and electrode pairs for inducing sexual climax by the user; and wherein, in use, an intensity of at least one of an output of the vibrating device and electrical impulses are selectively varied during the use.
